(19) United States
(12) Patent Application Publication
(10) Pub. No.: US 2006/0122425 A1
(43) Pub. Date: Jun. 8, 2006

(54) Method for the production of a dicarboxylic acid from acrylic acid

(76) Inventors: Peter Bassler, Viernheim (DE); Stefan Maixner, Schwetzingen (DE); Jens Scheidel, Hirschberg (DE)

Correspondence Address: CONNOLLY BOVE LODGE & HUTZ, LLP, P O BOX 2207, WILMINGTON, DE, 19899, US

(21) Appl. No.: 10/519,523

(22) PCT Filed: Jul. 4, 2003
(86) PCT No.: PCT/EP03/07151

§ 371 Date: Dec. 27, 2004

(30) Foreign Application Priority Data
Jul. 10, 2002 (DE) 102 31 291.5
Aug. 30, 2002 (DE) 102 40 781.9

(51) Int. Cl. C07C 51/347

(52) U.S. Cl. 562/590

(57) ABSTRACT

A process for the preparation of a dicarboxylic acid of the formula (I)
—H—OOC-(n-C₄Hₓ)—COO—H (I) where x is 6 or 8, starting from acrylic acid, which comprises a) reacting a dicarboxylic acid diester of the formula (II)
R¹—OOC-(n-C₄Hₓ)—COO—R² (II) where x is 6 or 8, and
R¹ and R², independently of one another, are C₁-, C₂-, C₃- or C₄-alkyl, aryl or heteroaryl and may be identical to or different from one another,
with acrylic acid to give a dicarboxylic acid of the formula (I) and a mixture of acrylic acid esters of the formulae C₂H₃—COOR¹ and C₂H₃—COOR², where R¹ and R² are as defined above, b) separating the dicarboxylic acid of the formula (I) obtained in step a) from the reaction mixture obtained in step a),

c) dimerizing the C₂H₃—COOR¹, C₂H₃—COOR² or mixture thereof obtained in step a) to give an n-butenedicarboxylic acid diester, and d) cleaving the dicarboxylic acid diester obtained in step c) into the corresponding dicarboxylic acid of the formula (I).

The present invention relates to a process for the preparation of a dicarboxylic acid of the formula (I)
H—OOC-(n-C₄Hₓ)—COO—H (I)

where

x is 6 or 8,

starting from acrylic acid,

which comprises

a) reacting a dicarboxylic acid diester of the formula (II)
R¹—OOC-(n-C₄Hₓ)—COO—R² (II)

where

x is 6 or 8, and

R¹ and R², independently of one another, are C₁-, C₂-, C₃- or C₄-alkyl, aryl or heteroaryl and may be identical to or different from one another,

with acrylic acid to give a dicarboxylic acid of the formula (I) and a mixture of acrylic acid esters of the formulae C₂H₃—COOR¹ and C₂H₃—COOR², where R¹ and R² are as defined above,

b) separating the dicarboxylic acid of the formula (I) obtained in step a) from the reaction mixture obtained in step a),

c) dimerizing the C₂H₃—COOR¹, C₂H₃—COOR² or mixture thereof obtained in step a) to give an n-butenedicarboxylic acid diester, and

d) cleaving the dicarboxylic acid diester obtained in step c) into the corresponding dicarboxylic acid of the formula (I).

Processes for the preparation of dimethyl n-butenedicarboxylate, i.e. dicarboxylic acid diesters (II) where x=6 and R¹=R²=methyl, starting from methyl acrylate are known per se.

Thus, U.S. Pat. No. 3,013,066 describes in Examples XX and XXI the dimerization of methyl acrylate in the presence of ruthenium chloride as catalyst., Dimethyl n-butenedicarboxylate is obtained in Example XX as fraction II in a yield of only 24% and in Example XXI as fraction III in a yield of only 37%, in each case based on methyl acrylate employed.

U.S. Pat. No. 4,638,084 describes in Example I the dimerization of methyl acrylate in the presence of chlorobis(ethylene)rhodium(I) dimer and silver tetrafluoroborate as catalyst. At a conversion of 100%, dimethyl n-butenedicarboxylate was obtained in a yield of only 60%, based on methyl acrylate employed, determined by NMR.

EP-A-475 386 describes the dimerization of methyl acrylate in the presence of specific rhodium complexes as catalyst. According to Example 4, a conversion of 97% to dimethyl n-butenedicarboxylate is achieved, determined by NMR.

However, it is usually not dimethyl n-butenedicarboxylate that is in demand as an industrially important product, but instead a dicarboxylic acid (I), in particular adipic acid, i.e. a dicarboxylic acid (I) where x=6. Adipic acid is an important intermediate in the preparation of polymer plasticizers, of polyesterols, for example for polyurethanes, and a starting monomer for the preparation of industrially important polymers, such as nylon 6,6.

According to U.S. Pat. No. 3,013,066, Examples XX and XXI, the dimethyl n-butenedicarboxylate obtained in the dimerization can subsequently be hydrogenated to dimethyl adipate after removal from a product mixture, and adipic acid can be obtained by saponification of the adipic acid diester.

The process described in U.S. Pat. No. 3,013,066 for the preparation of adipic acid starting from methyl acrylate thus disadvantageously includes a multiplicity of process steps for the preparation of four intermediates, namely acrylic acid, methyl acrylate, dimethyl n-butenedicarboxylate and dimethyl adipate, where it should be taken into account that in addition to the acrylic acid esterification and the hydrogenation of the dimethyl n-butenedicarboxylate to dimethyl adipate, a separation step is likewise necessary, such as the removal of the resultant adipic acid from the product mixture after the saponification of the dimethyl adipate.

In addition, as is known, the methyl acrylate employed for dimerization in the process described must firstly be prepared by esterification of acrylic acid, where at least one separation step is likewise necessary in order to obtain the ester in pure form.

It is an object of the present invention to provide a process which enables the preparation of a dicarboxylic acid (I), in particular adipic acid, from acrylic acid in a technically simple and economical manner.

We have found that this object is achieved by the process defined at the outset.

In accordance with the invention, acrylic acid is reacted in step a) with a dicarboxylic acid diester of the formula (II)
R¹—OOC-(n-C₄Hₓ)—COO—R² (II)

where

x is 6 or 8.

In the formula (II), R¹ and R², independently of one another, are C₁-, C₂-, C₃- or C₄-alkyl, such as methyl, ethyl, n-propyl, i-propyl, n-butyl, i-butyl, s-butyl or t-butyl, preferably methyl, aryl, such as phenyl, or heteroaryl. R¹ and R² are preferably, independently of one another, C₁-, C₂-, C₃- or C₄-alkyl, such as methyl, ethyl, n-propyl, i-propyl, n-butyl, i-butyl, s-butyl or t-butyl, in particular methyl.

R¹ and R² may be different. In a preferred embodiment, R¹ and R² are identical. In a particularly preferred embodiment, R¹ and R² are identical and are both methyl.

In the case where x=8, the dicarboxylic acid on which the dicarboxylic acid ester of the formula (II) is based is adipic acid.

The corresponding adipic acid diesters of the formula (II) and their preparation are known per se. Thus, the adipic acid diesters can be obtained, for example, by dicarbonylation of butadiene in the presence of alcohols, such as methanol.

In a preferred embodiment, the butenedicarboxylic acid ester obtained in step c) of the process according to the invention can be hydrogenated to an adipic acid diester. This hydrogenation can be carried out in a manner known per se, for example with homogeneous or heterogeneous, preferably heterogeneous catalysis.

Suitable heterogeneous catalysts are preferably those which contain, as catalytically active components, a noble metal from group 8 of the Periodic Table of the Elements, such as palladium, ruthenium, rhodium, iridium, platinum, nickel, cobalt or copper, preferably palladium.

These metals can be employed in unsupported form, for example as suspension catalysts, preferably in the case of nickel or cobalt.

These metals can be employed in supported form, for example on activated carbon, metal oxides, transition-metal oxides, in particular aluminum oxide or silicon oxide, preferably as fixed-bed catalysts.

The adipic acid diester obtained in this hydrogenation can advantageously be employed in step a).

In the case where x=6, the dicarboxylic acid on which the dicarboxylic acid ester of the formula (II) is based is n-butenedicarboxylic acid or a mixture of isomeric n-butenedicarboxylic acid esters.

The corresponding n-butenedicarboxylic acid diesters of the formula (II) and their preparation are known per se. Thus, the n-butenecarboxylic acid diesters can be obtained, for example, by dimerization of acrylic acid esters, as described in U.S. Pat. No. 3,013,066, U.S. Pat. No. 4,638,084 or EP-A-475 386 mentioned at the outset or as also described below in J. Am. Chem. Soc. 87 (1965) 5638-5645 or J. Molecular Catalysis 29 (1985) 65-76 or step c) according to the invention.

The acrylic acid employed in step a) and processes for the preparation thereof are known. Thus, for example, acrylic acid can be obtained by gas-phase oxidation of propene or propane in the presence of heterogeneous catalysts.

When acrylic acid is stored or worked up, it is customary to add one or more stabilizers which, for example, prevent or reduce the polymerization or decomposition of acrylic acid, such as p-methoxyphenol or 4-hydroxy-2,2,4,4-piperidine N-oxyl (“4-hydroxy-TEMPO”).

Before the acrylic acid is used in the process according to the invention, some or all of such stabilizers may be removed. The stabilizers may be removed by processes known per se for this purpose, such as distillation, extraction or crystallization.

Such stabilizers may remain in the acrylic acid in the abovementioned amount.

Furthermore, acrylic acid esters can be obtained, for example, by esterification of acrylic acid with the corresponding alcohols in the presence of homogeneous catalysts, such as p-toluenesulfonic acid.

The reaction of the dicarboxylic acid diester of the formula (II) with acrylic acid can be carried out without catalysis.

In an advantageous embodiment, it is possible to use a homogeneous or heterogeneous catalyst, in particular a heterogeneous catalyst. The catalyst employed can preferably be an inorganic or organic, Lewis or Brönstedt acid compound. In the case of organic compounds, ion exchangers can advantageously be used. In the case of inorganic compounds, oxides having acidic centers, such as zeolites, are advantageously suitable.

In a further advantageous embodiment, the use of a homogeneous catalyst together with a heterogeneous catalyst is considered. Preferred catalysts are inorganic or organic, Lewis or Brönsted acid compounds.

In the case of organic compounds, advantageous heterogeneous catalysts are ion exchangers; in the case of inorganic compounds, advantageous compounds are oxides having acidic centers, such as zeolites.

In the case of organic compounds, an advantageous homogeneous catalyst is p-toluenesulfonic acid; in the case of inorganic compounds, advantageous compounds are sulfuric acid or phosphoric acid.

Homogeneous and heterogeneous catalysts may be used at the same time or in succession, such as first the homogeneous and then the heterogeneous catalyst, or first the heterogeneous and then the homogeneous catalyst.

In a further advantageous embodiment, the use of a homogeneous catalyst is considered. Preferred catalysts are inorganic or organic, Lewis or Brönsted acid compounds.

In the case of organic compounds, p-toluenesulfonic acid may advantageously be used; in the case of inorganic compounds, advantageous compounds are sulfuric acid or phosphoric acid.

The reaction in step a) can be carried out in a reactor, such as a stirred reactor, a reactor cascade, such as a stirred-reactor cascade, or in a distillation device, preferably in one having a reaction vessel, advantageously in a reactive distillation column, in particular one having a dividing wall.

If the reaction is carried out in a distillation device, the catalyst, in the case of reaction in step a) in the presence of a catalyst, can advantageously be installed in the region between the bottom and top of the distillation device.

In step a), a reaction mixture is obtained which comprises a dicarboxylic acid of the formula (I) and a mixture of acrylic acid esters of the formulae C₂H₃—COOR¹ and C₂H₃—COOR², where R¹ and R² are as defined above. The reaction mixture may furthermore comprise dicarboxylic acid diesters of the formula (II), acrylic acid, dicarboxylic acid monoesters of the formula. R¹—OOC-(n-C₄Hₓ)—COOH or HOOC-(n-C₄Hₓ)—COO—R² , where R¹, R² and x are as defined above, R¹OH, R²OH, water or mixtures thereof.

In step b) according to the invention, the dicarboxylic acid of the formula (I) obtained is separated off from the reaction mixture obtained in step a).

The separation in step b) can be carried out in a step which is separate from step a). If, for example, one of the reactors mentioned or one of the reactor cascades mentioned is employed in step a), the product mixture can be withdrawn from the reactor or the final reactor of the reactor cascade, and the dicarboxylic acid of the formula (I) can subsequently be separated off from the reaction mixture obtained in step a) by separation operations known per se, such as distillation, extraction or crystallization, in one or more steps.

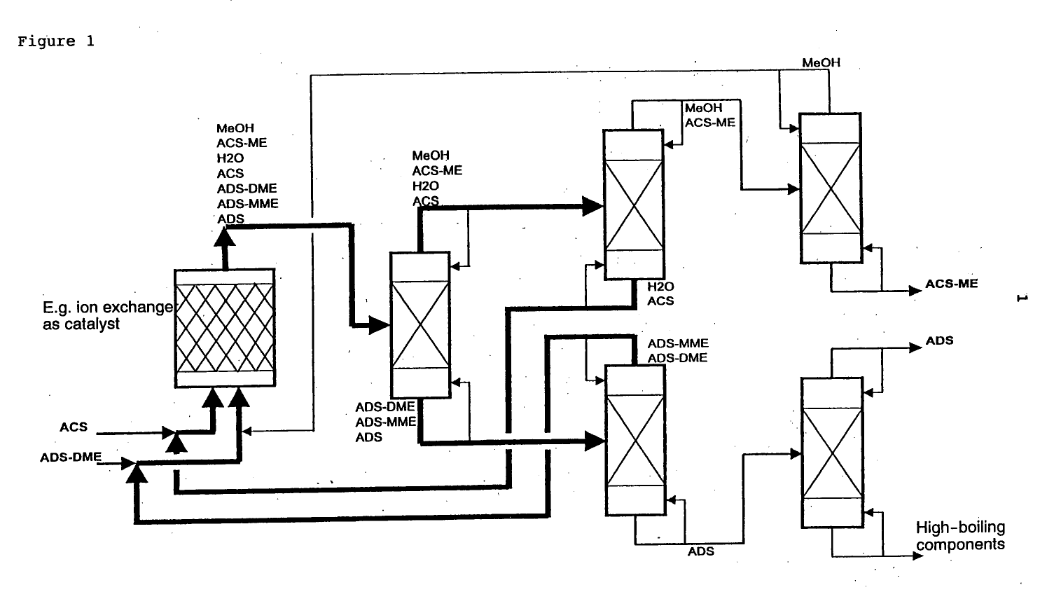

A process of this type is depicted diagrammatically in FIG. 1 with reference to the example of the reaction of dimethyl adipate with acrylic acid. In the drawing, the abbreviations have the following meanings:
MeOH: methanol
ACS: acrylic acid
ACS-ME: methyl acrylate
ADS: adipic acid
ADS-MME: monomethyl adipate
ADS-DME: diumethyl adipate
H2O: water
Cross-hatched area: optional catalyst

In an advantageous embodiment, steps a) and b) can be carried out together in part or in their entirety. The reaction in step a) in a distillation device is preferably suitable here.

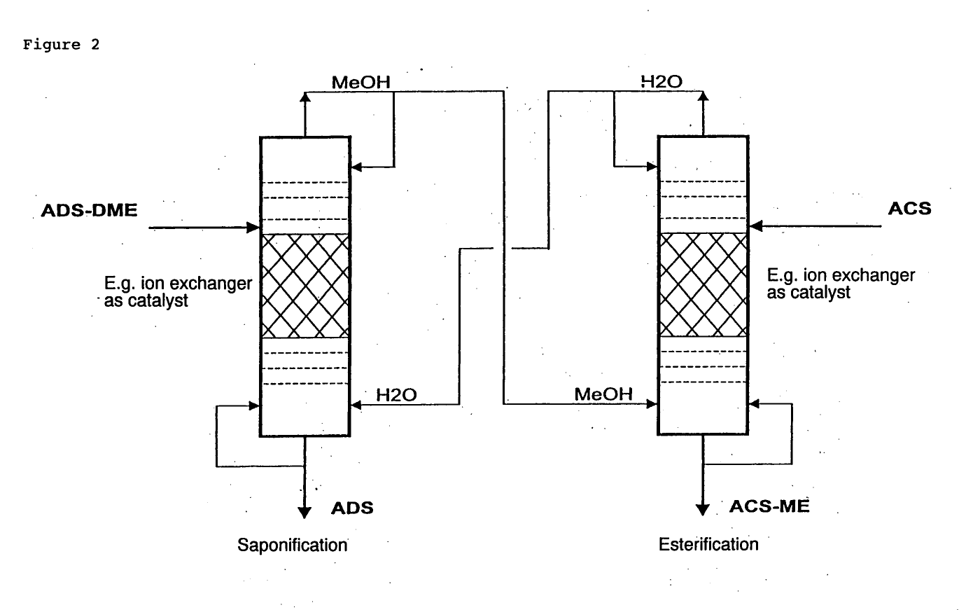

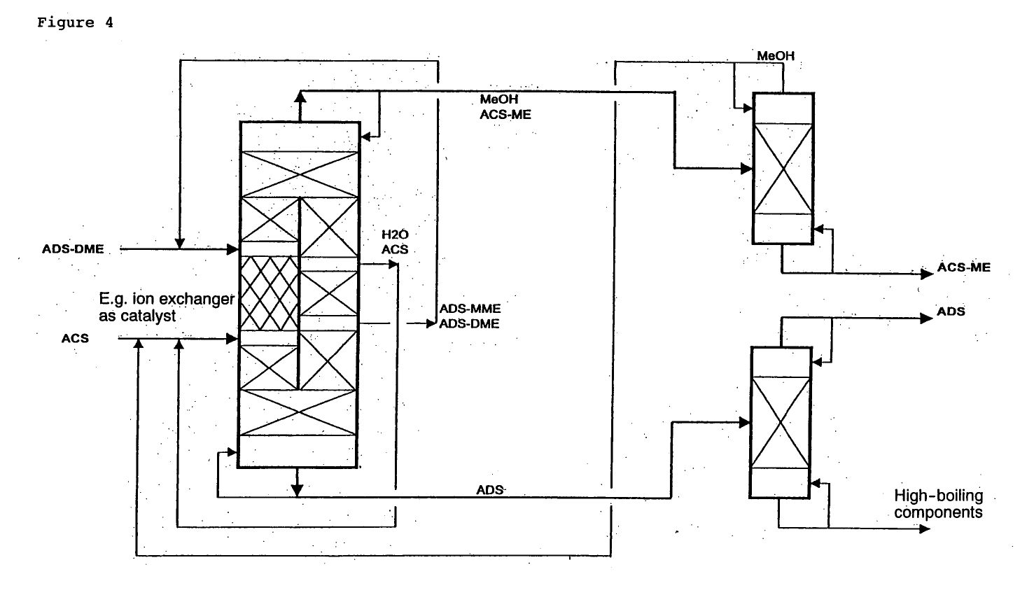

In an advantageous embodiment, the distillation device can be operated in such a way that the dicarboxylic acid is obtained as a component which is separate from the remainder of the reaction mixture. This is shown diagrammatically in FIGS. 2 and 4, again depicted by way of example with reference to the reaction of dimethyl adipate with acrylic acid, where the abbreviations have the above-mentioned meanings.

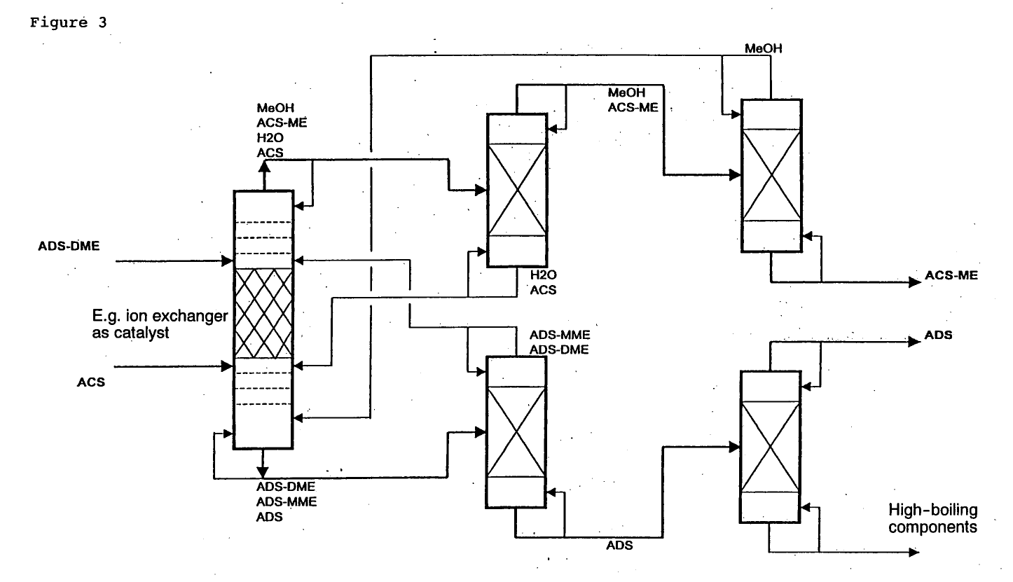

In a further advantageous embodiment, the distillation device can be operated in such a way that the dicarboxylic acid and at least one of its esters, i.e. dicarboxylic acid monoesters, dicarboxylic acid diesters or mixtures thereof, is obtained as a component which is separate from the remainder of the reaction mixture, and the dicarboxylic acid is subsequently separated off from this mixture. This is shown diagrammatically in FIG. 3, again depicted by way of example with reference to the reaction of dimethyl adipate with acrylic acid, where the abbreviations have the above-mentioned meanings.

In the case where x=8, adipic acid can be obtained from step b).

In the case where x=6, n-butenedicarboxylic acid can be obtained from step b).

In a preferred embodiment, the butenedicarboxylic acid obtained in step b) of the process according to the invention can be hydrogenated to adipic acid. This hydrogenation can be carried out in a manner known per se, for example with homogeneous or heterogeneous, preferably heterogeneous catalysis.

Suitable heterogeneous catalysts are preferably those which contain, as catalytically active component, a noble metal from group 8 of the Periodic Table of the Elements, such as palladium, ruthenium, rhodium, iridium, platinum, nickel, cobalt or copper, preferably palladium.

These metals can be employed in unsupported form, for example as suspension catalysts, preferably in the case of nickel or cobalt.

These metals can be employed in supported form, for example on activated carbon, metal oxides, transition-metal oxides, in particular aluminum oxide, or silicon oxide, preferably as fixed-bed catalysts.

In accordance with the invention, the acrylic esters C₂H₃—COOR¹ and C₂H₃—COOR² or mixtures thereof obtained in step a) are dimerized in step c) to give n-butenedicarboxylic acid diesters.

Processes for the dimerization of acrylic esters to give n-butenedicarboxylic acid esters are known per se.

Thus, the dimerization can advantageously be carried out in the presence of a catalyst.

In an advantageous embodiment, it is possible to use a homogeneous catalyst, such as a catalyst containing an element from group 8 of the Periodic Table of the Elements, in particular rhodium or ruthenium, preferably in the form of a salt, such as a chloride, or a complex compound. Catalysts of this type and processes for the dimerization of acrylic esters to give n-butenedicarboxylic acid diesters in the presence of catalysts of this type are described, for example, in U.S. Pat. No. 3,013,066, U.S. Pat. No. 4,638,084.and EP-A-475 386 mentioned at the outset or also in J. Am. Chem. Soc. 87 (1965) 5638-5645 or J. Molecular Catalysis 29 (1985) 65-76.

The dicarboxylic acid ester obtained in step c) is, in accordance with the invention, cleaved into the corresponding dicarboxylic acid of the formula (I).

Processes for the cleavage of an ester to give the corresponding carboxylic acid are known per se, for example from U.S. Pat. No. 5,710,325 or U.S. Pat. No. 5,840,959.

In the case where x=6, the n-butenedicarboxylic acid obtained in step d) can advantageously be hydrogenated to give adipic acid.

This hydrogenation can be carried out in a manner known per se, for example with homogeneous or heterogeneous, preferably heterogeneous catalysis.

Suitable heterogeneous catalysts are preferably those which contain, as catalytically active component, a noble metal from group 8 of the Periodic Table of the Elements, such as palladium, ruthenium, rhodium, iridium, platinum, nickel, cobalt or copper, preferably palladium.

These metals can be employed in unsupported form, for example as suspension catalysts, preferably in the case of nickel or cobalt.

These metals can be employed in supported form, for example on activated carbon, metal oxides, transition-metal oxides, in particular aluminum oxide, or silicon dioxide, preferably as fixed-bed catalysts.

In an advantageous embodiment, the cleavage of n-butenedicarboxylic acid diesters in step d) can, in the case where x=6, be carried out by recycling the n-butenedicarboxylic acid ester obtained in step c) into step a), converting this n-butenedicarboxylic acid diester into n-butenedicarboxylic acid in step a), and obtaining n-butenedicarboxylic acid in step b) as the dicarboxylic acid of the formula (I).

The n-butenedicarboxylic acid obtained in step d) can particularly advantageously be hydrogenated here to give adipic acid.

This hydrogenation can be carried out in a manner known per se, for example with homogeneous or heterogeneous, preferably heterogeneous catalysis.

Suitable heterogeneous catalysts are preferably those which contain, as catalytically active component, a noble metal from group 8 of the Periodic Table of the Elements, such as palladium, ruthenium, rhodium, iridium, platinum, nickel, cobalt or copper, preferably palladium.

These metals can be employed in unsupported form, for example as suspension catalysts, preferably in the case of nickel or cobalt.

These metals can be employed in supported form, for example on activated carbon, metal oxides, transition-metal oxides, in particular aluminum oxide, or silicon dioxide, preferably as fixed-bed catalysts.

In a further preferred embodiment, the n-butenedicarboxylic acid diester obtained in step c) in the case where x=6 can be hydrogenated between steps c) and d) to give an adipic acid diester.

This hydrogenation can be carried out in a manner known per se, for example with homogeneous or heterogeneous, preferably heterogeneous catalysis.

Suitable heterogeneous catalysts are preferably those which contain, as catalytically active component, a noble metal from group 8 of the Periodic Table of the Elements, such as palladium, ruthenium, rhodium, iridium, platinum, nickel, cobalt or copper, preferably palladium.

These metals can be employed in unsupported form, for example as suspension catalysts, preferably in the case of nickel or cobalt.

These metals can be employed in supported form, for example on activated carbon, metal oxides, transition-metal oxides, in particular aluminum oxide, or silicon dioxide, preferably as fixed-bed catalysts.

Adipic acid can be obtained by cleavage of the adipic acid diester in step d).

In an advantageous embodiment, the cleavage of the adipic acid diester in step d) can be carried out by recycling the resultant adipic acid diester into step a), converting this adipic acid diester into adipic acid in step a), and obtaining adipic acid as the dicarboxylic acid of the formula (I) in step b).

The azeotropes which can arise in the distillative separations carried out in the process according to the invention can result in changes to the said material streams which are insignificant and merely slight for the purposes of the present invention. The separation of such azeotropes to give the substances mentioned in the steps according to the invention can be carried out by methods known per se.

### EXAMPLES

In examples 1-3, the reaction vessel used was a three-neck round-bottom flask of capacity 500 ml which was purged with nitrogen before the start of the experiment. The mixture was heated to the particular temperature with stirring. A heated line led from one outlet of the flask to a cold trap cooled by dry ice. Downstream of the cold trap was a controlled vacuum pump protected by a reflux trap.

### Example 1

In the cold trap, 21.22 g of methyl acrylate and 0.13 g of 4-hydroxy-TEMPO were initially charged. In the reaction vessel, a mixture of 21.80 g of dimethyl adipate, 36.03 g of acrylic acid and 0.36 g of 4-hydroxy-TEMPO was heated to 50° C. 0.23 g of concentrated sulfuric acid was then added (t =0 h) and the pressure in the reaction vessel was regulated to 10 kPa.

After 24 hours (t=24 h),.samples were taken from the reaction vessel and the cold trap and analyzed by means of an HP 5890 gas chromatograph having an HP5 column. At the same time, the reaction temperature was increased to 65° C. at unchanged pressure. After 45 hours (t=45 h), a sample was taken from the reaction vessel, and after 65 hours (t=65 h), further samples were taken from the reaction vessel and the cooling trap and likewise analyzed.

In table 1, the proportions by weight in percent based on the sum of the weights of the five components specified in each sample are reported.

TABLE 1

| | Time | | | | | |
|---|---|---|---|---|---|---|
| | | | 24 h | | | 65 h |
| | 0 h | 24 h | Cold | 45 h | 65 h | Cold |
| Sample | Reactor | Reactor | trap | Reactor | Reactor | trap |
| MAC | 0.0 | 1.0 | 100.0 | 0.9 | 0.0 | 5.1 |
| ACA | 47.8 | 44.2 | 0.0 | 41.6 | 30.9 | 94.9 |
| DMAD | 50.7 | 49.5 | 0.0 | 48.9 | 53.8 | 0.0 |
| MMAD | 1.5 | 5.0 | 0.0 | 8.1 | 14.2 | 0.0 |
| ADA | 0.0 | 0.3 | 0.0 | 0.6 | 1.2 | 0.0 |

The abbreviations are defined as follows:
MAC methyl acrylate
ACA acrylic acid
DMAD dimethyl adipate
MMAD monomethyl adipate
ADA adipic acid

### Example 2

In the reaction vessel, a mixture of 21.78 g of dimethyl adipate, 36.03 g of acrylic acid, 0.13 g of methanol and 0.37 g of 4-hydroxy-TEMPO was heated to 65° C. 5.01 g of Lewatit S100 G1 were then added in the H⁺ form (t=0 h) and the pressures in the reaction vessel was regulated at 10 kPa.

After 24 hours (t=24 h), a sample was taken from the reaction vessel and analyzed according to example 1. At the same time, 0.13 g of concentrated sulfuric acid were added at unchanged pressure. After 48 hours (t=48 h), samples were taken from the reaction vessel and the cold trap and likewise analyzed.

In table 2, the proportions by weight in percent based on the sum of the weights of the five components specified in each sample are reported.

TABLE 2

Time

| | 0 h | 24 h | 48 h | 48 h |
|---|---|---|---|---|
| Sample | Reactor | Reactor | Reactor | Cold trap |
| MAC | 0.0 | 0.0 | 4.2 | 83.1 |
| ACA | 48.1 | 44.6 | 35.8 | 16.3 |
| DMAD | 50.4 | 52.1 | 22.2 | 0.0 |
| MMAD | 1.5 | 2.9 | 28.6 | 0.6 |
| ADA | 0.0 | 0.4 | 9.3 | 0.0 |

### Example 3

In the reaction vessel, a mixture of 20.03 g of monomethyl adipate, 36.03 g of acrylic acid, 0.14 g of methanol and 0.35 g of 4-hydroxy-TEMPO was heated to 65° C. 0.24 g of concentrated sulfuric acid was then added (t=0 h) and the pressure in the reaction vessel was regulated at 10 kPa.

After 24 hours (t=24 h) a sample was taken from the reaction vessel and analyzed according to example 1. After 50 hours (t=50 h), samples were taken from the reaction vessel and the cold trap and likewise analyzed.

In table 3, the proportions by weight in percent based on the sum of the weights of the five components specified in each sample are reported.

TABLE 3

Time

| | 0 h | 25 h | 50 h | 50 h |
|---|---|---|---|---|
| Sample | Reactor | Reactor | Reactor | Cold trap |
| MAC | 0.0 | 1.2 | 1.5 | 42.1 |
| ACA | 50.5 | 46.2 | 43.7 | 57.8 |
| DMAD | 0.0 | 10.5 | 12.1 | 0.0 |
| MMAD | 47.9 | 32.0 | 28.3 | 0.0 |
| ADA | 1.6 | 10.1 | 14.5 | 0.0 |

1. A process for the preparation of a dicarboxylic acid of the formula (I)
H—OOC-(n-C₄Hₓ)—COO—H (I)
where
x is 6 or 8,
starting from acrylic acid,
which comprises
a) reacting a dicarboxylic acid diester of the formula (II)
R¹—OOC-(n-C₄Hₓ)—COO—R² (II)
where
x is 6 or 8, and
R¹ and R², independently of one another, are C₁-, C₂-, C₃- or C₄-alkyl, aryl or heteroaryl and may be identical to or different from one another,
with acrylic acid to give a dicarboxylic acid of the formula (I) and a mixture of acrylic acid esters of the formulae C₂H₃—COOR¹ and C₂H₃—COOR², where R¹ and R² are as defined above,
b) separating the dicarboxylic acid of the formula (I) obtained in step a) from the reaction mixture obtained in step a),
c) dimerizing the C₂H₃—COOR¹, C₂H₃—COOR² or mixture thereof obtained in step a) to give an n-butenedicarboxylic acid diester, and
d) cleaving the dicarboxylic acid diester obtained in step c) to give the corresponding dicarboxylic acid of the formula (I).

2. A process as claimed in claim 1, where the cleavage of the n-butenedicarboxylic acid diester in step d) is carried out by
recycling the n-butenedicarboxylic acid ester obtained in step c) into step a), converting this n-butenedicarboxylic acid diester into n-butenedicarboxylic acid in step a), and
obtaining n-butenedicarboxylic acid as the dicarboxylic acid of the formula (I) in step b).

3. A process as claimed in claim 1, where the n-butenedicarboxylic acid obtained in step d) is hydrogenated to give adipic acid as the dicarboxylic acid of the formula (I).

4. A process as claimed in claim 1, where the cleavage of the n-butenedicarboxylic acid diester in step d) is carried out by
recycling the n-butenedicarboxylic acid ester obtained in step c) into step a),
converting this n-butenedicarboxylic acid diester into n-butenedicarboxylic acid in step a),
obtaining n-butenedicarboxylic acid in step b), and
hydrogenating this n-butenedicarboxylic acid to give adipic acid as the dicarboxylic acid of the formula (I).

5. A process as claimed in claim 1, where
the n-butenedicarboxylic acid diester obtained in step c) is hydrogenated between steps c) and d) to give an adipic acid diester, and
adipic acid is obtained as the dicarboxylic acid of the formula (I) by cleaving the adipic acid diester in step d).

6. A process as claimed in claim 1, where
the n-butenedicarboxylic acid diester obtained in step c) is hydrogenated between steps c) and d) to give an adipic acid diester,
the cleavage of the adipic acid diester in step d) is carried out by recycling the resultant adipic acid diester into step a) and converting it into adipic acid in step a), and
adipic acid is obtained as the dicarboxylic acid of the formula (I) in step b).

7. A process as claimed in claim 1, where the radicals R¹ and R² are, independently of one another, methyl, ethyl, n-propyl, i-propyl, n-butyl, i-butyl, s-butyl or t-butyl.

8. A process as claimed in claim 1, where the radicals R¹ and R² are identical.

9. A process as claimed in claim 1, wherein the radicals R¹ and R² are methyl.

10. A process as claimed in claim 1, wherein x=8, and the dicarboxylic acid on which the dicarboxylic acid ester of the formula (II) is based is adipic acid.

11. A process as claimed in claim 5, wherein the hydrogenation is carried out with a heterogeneous catalyst.

12. A process as claimed in claim 11, wherein said heterogeneous catalyst comprise a noble metal from group 8 of the Periodic Table of the Elements.

13. A process as claimed in claim 12, wherein said heterogeneous catalyst comprise palladium, ruthenium, rhodium, iridium, platinum, nickel, cobalt or copper.

14. A process as claimed in claim 1, wherein x=8, and the adipic acid is obtained from step b).

15. A process as claimed in claim 1, wherein x=6, and n-butenedicaboxylic acid is obtained from step b).
